(19) United States
(12) Patent Application Publication
(10) Pub. No.: US 2024/0197595 A1
(43) Pub. Date: Jun. 20, 2024

(54) RESHAPING COMPOSITION FOR KERATIN FIBERS

(71) Applicant: KAO GERMANY GMBH, Darmstadt (DE)

(72) Inventors: Ruth KAMGUIA, Darmstadt (DE); Sabine SCHAEFER, Darmstadt (DE); Normen LIPINSKI, Darmstadt (DE)

(73) Assignee: KAO GERMANY GMBH, Darmstadt (DE)

(21) Appl. No.: 18/286,890

(22) PCT Filed: Apr. 19, 2022
(86) PCT No.: PCT/EP2022/060291

§ 371 Date: Oct. 13, 2023

(30) Foreign Application Priority Data
Apr. 26, 2021 (EP) 21170414.3

(51) Int. Cl. A61K 8/41; A61Q 5/04

(52) U.S. Cl. CPC A61K 8/41; A61Q 5/04; A61K 2800/48; A61K 2800/81; A61K 2800/88

(57) ABSTRACT

Cosmetic compositions for keratin fibers, kits-of-parts comprising the cosmetic compositions, and keratin fiber reshaping processes are disclosed herein. The cosmetic compositions have a pH in the range of 7 to 12 and comprise one or more organic alkalizing agent(s) and one or more particulate thickening agent(s), wherein the cosmetic compositions comprise less than 1% by weight of oxidizing agents, calculated to the total weights of the cosmetic compositions.

### FIELD OF THE INVENTION

The present invention is directed to a reshaping composition for keratin fibers, a reshaping process, and kit-of-parts.

### BACKGROUND OF THE INVENTION

Current permanent reshaping processes require steps of reducing the hair and later oxidizing the hair. The reducing step employs reducing agents, such as thioglycolic acid, in order to cleave disulphide bonds within the hair and to allow protein chain movement. In a subsequent step the bonds are reformed by addition of an oxidizing composition, typically comprising hydrogen peroxide. In Western countries these well known processes include cold perming and hot perming techniques, whereas, especially in Asian countries, a digital perming as a specialty of the hot perming process is preferred. For a digital perming process the temperature of the perm is controlled by an electronic device, often being equipped with a microprocessor. These multi-step processes are time-consuming (up to 3 hours for a cold perm/5 hours fora digital perm), require a high level of winding skill from the stylist, cause hair damage and, at worst, can lead to hair loss due to over-processing. Over-processing typically stems from either leaving the reducing agent for too long on the hair, or heating the hair to inappropriate temperatures for a too long time. To avoid over-processing, a test curler is typically carried out on a streak of the client's hair prior to the full process being performed, in order to determine the optimum processing time; this adds further to the time needed and requires further skills/education from the stylist. Apart from all the process challenges, the reducing composition has a strong and acrid odour disliked by stylists and consumers alike.

For easing the application of reshaping compositions, WO2016/098870 discloses alkaline non-reducing, non-oxidizing compositions comprising fatty compounds and water-soluble thickening polymers.

However, stability and performance of these compositions still remain challenging.

### SUMMARY OF THE INVENTION

The first object of the present invention is a cosmetic composition for reshaping keratin fibers with heat, preferably for reshaping human keratin fibers with heat, more preferably for reshaping human hair with heat, having a pH in the range of 7 to 12, and comprising:
a) one or more alkalizing agent(s), preferably selected from
   ammonia and/or ammonium salt(s), and/or
   organic amine(s) and/or salt(s) of organic amines according to the following general structure:

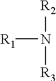

wherein R₁, R₂, and R₃ are independently selected from H, linear C₁-C₆ alkyl which may be substituted with one hydroxyl group, or branched C₃-C₁₂alkyl or alkanol, wherein at least one of R₁, R₂, or R₃ is different from H, and/or
   mixtures of the alkalizing agents from above,
b) one or more particulate thickening agent(s),
wherein the composition comprises less than 1% by weight of oxidizing agents, calculated to the total weight of the composition.

Preferably, from the viewpoint of cosmetic application the composition is a permanent reshaping composition for reshaping with heat, preferably with heat above 50° C., more preferably with heat at 80° C. or above. Still more preferably the composition is a permanent waving composition.

The second object of the present invention is a process for reshaping keratin fibers, preferably human keratin fibers, more preferably human hair comprising the steps of:
i) putting keratin fibers under mechanical tension,
ii) applying to keratin fibers the composition as defined above,
iii) optionally covering keratin fibers with a moisture barrier,
iv) heating the keratin fibers to a temperature in the range of 50° C. to 230° C.,
v) optionally removing the moisture barrier from keratin fibers,
vi) releasing tension from keratin fibers,
vii) optionally rinsing-off the keratin fibers,
wherein process steps i), ii), and vi), vii) can be executed in either order.

The third object of the present invention is a kit-of-parts comprising
a composition A having a pH in the range of 7 to 12 and comprising one or more compound(s) according to group a) as defined above,
a composition B comprising one or more compound(s) according to group b) as defined above.

The compositions of the kit of the third object of the invention are preferably kept separate until directly prior to application onto keratin fibers.

The fourth object of the present invention is a kit-of-parts comprising the composition as defined for the first object of the present invention and one or more digital perm curler(s).

The fifth object of the present invention is the use of the composition as defined above for permanent reshaping of keratin fibers, preferably of human keratin fibers, more preferably of human hair, still more preferably for permanent waving of human hair.

### DETAILED DESCRIPTION OF THE INVENTION

Inventors of the present invention have unexpectedly found out that a composition according to claim 1 has excellent heat stability during perming, high curling efficiency, improved cosmetic safety by preventing dripping at process temperatures above 50° C., and confers cosmetic properties to keratin fibers such as feel, touch, and shine. Moreover, the composition has less to no odor and may be applied without having to cover the keratin fibers with a moisture barrier.

### Reshaping Composition

The present invention is directed to a cosmetic composition for reshaping keratin fibers with heat, preferably for reshaping human keratin fibers with heat, more preferably for reshaping human hair with heat, having a pH in the range of 7 to 12, and comprising:
a) one or more alkalizing agent(s), preferably selected from
   ammonia and/or ammonium salt(s), and/or
   organic amine(s) and/or salt(s) of organic amines according to the following general structure:

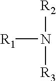

wherein R₁, R₂, and R₃ are independently selected from H, linear C₁-C₆ alkyl which may be substituted with one hydroxyl group, or branched C₃-C₁₂alkyl or alkanol, wherein at least one of R₁, R₂, or R₃ is different from H, and/or
   mixtures of the alkalizing agents from above,
b) one or more particulate thickening agent(s),
wherein the composition comprises less than 1% by weight of oxidizing agents, calculated to the total weight of the composition.

The term ‘particulate’ within the meaning of the present invention denotes a compound in solid form at 25° C. and atmospheric pressure. This compound is partially or completely insoluble with water, but may hydrate to a certain degree to thicken aqueous compositions. To qualify as particulate thickening agent, it is preferred to increase the viscosity of water at a concentration of 1% by weight of the particulate thickening agent to 100 mPas or more, measured by cone-plate viscometry with a Brookfield viscometer and spindle #5.

Preferably, one or more compound(s) according to group b) has a solubility in water at 25° C. and atmospheric pressure of 1 g/L or less, preferably of 0.5 g/L or less, more preferably of 0.1 g/L or less, determined by placing an aqueous dispersion of one or more compound(s) according to group b) in a glass vial, shaking it for 24 h at 25° C., and gravimetrically determining the dissolved amount in the supernatant after evaporating the solvent.

The composition of present invention may comprise low amounts of reducing agents for stabilizing purposes. Preferably, the composition comprises less than 1% by weight of a reducing agent, preferably it comprises less than 0.1% by weight of a reducing agent, calculated to the total weight of the composition, further more preferably it is free of reducing agents.

The composition of present invention may comprise low amounts of oxidizing agents for stabilizing purposes. Preferably it comprises less than 0.1% by weight of an oxidizing agent, calculated to the total weight of the composition, further more preferably it is free of oxidizing agents.

It is further preferred from the viewpoint of reshaping durability that the composition from above is a composition for reshaping with heat at 50° C. or more, preferably with heat of 80° C. or more.

It is preferred from the viewpoint of keratin fiber reshaping performance that the pH of the composition is 7.5 or more, more preferably 8 or more, further more preferably 8.25 or more.

It is preferred from the viewpoint of keratin fiber reshaping performance, cosmetic safety, and damage to keratin fibers that the pH of the composition is 11 or less, more preferably 10.5 or less, further more preferably 10.0 or less.

For attaining the above-mentioned effects, it is preferred that the pH of the composition is in the range of 7.5 to 11, preferably in the range of 8.0 to 10.5, more preferably in the range of 8.25 to 10.

It is preferred from the viewpoint of rinsability that the composition of the present invention is an aqueous composition. In this respect, it is preferred that the composition of the present invention comprises water at 15% by weight or more, more preferably at 20% by weight or more, further more preferably at 30% by weight or more, still further more preferably at 40% by weight or more, still further more preferably at 50% by weight or more, still further more preferably at 60% by weight or more, still further more preferably at 80% by weight or more, calculated to the total weight of the composition.

It is preferred from the viewpoint of keratin fibers reshaping performance that the composition of the present invention comprises water at 90% by weight or less, more preferably at 85% by weight or less, further more preferably at 75% by weight or less, calculated to the total weight of the composition.

For attaining the above-mentioned effects, it is preferred that the composition of the present invention comprises water in the range of 15% to 90% by weight, more preferably 20% to 85% by weight, further more preferably 30% to 75% by weight, still further more preferably 40% to 75% by weight, still further more preferably 50% to 75% by weight, still further more preferably 60% to 75% by weight, calculated to the total weight of the composition.

The composition of the present invention may be an emulsion. Depending on the concentration of compound(s) according to group c), if present, it may be presented in the form of an oil-in-water emulsion. However, at higher concentrations of compound(s) according to group c), it may also be possible to formulate the composition as an inverse emulsion, i.e. a water-in-oil emulsion.

It is preferred from the viewpoint of hair damage that the composition of the present invention is free of bleaching agent(s) and/or oxidative dye(s).

### Alkalizing Agent(s) According to a)

The composition of the present invention comprises one or more alkalizing agent(s) as compound(s) according to group a), preferably selected from
ammonia and/or ammonium salt(s), and/or
organic amine(s) and/or salt(s) of organic amines according to the following general structure:

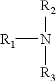

wherein R₁, R₂, and R₃ are independently selected from H, linear C₁-C₆ alkyl which may be substituted with one hydroxyl group, or branched C₃-C₁₂alkyl or alkanol, wherein at least one of R₁, R₂, or R₃ is different from H, and/or
mixtures of the alkalizing agents from above.

Suitable ammonium salts are inorganic ammonium and/or organic ammonium salts, and/or their mixtures. Inorganic ammonium salts are preferably selected from ammonium chloride, ammonium sulfate, ammonium carbonate, ammonium hydrogen carbonate, ammonium phosphates, ammonium hydrogen phosphates, ammonium dihydrogen phosphates, ammonium nitrate, ammonium bromide, ammonium iodide, ammonium thiosulfate, ammonium molybdate, ammonium vanadate, and/or their mixtures.

Suitable organic ammonium salts are preferably selected from ammonium carbamate, ammonium sulfamate, ammonium citrate, ammonium salicylate, ammonium valerate, ammonium tartarate, ammonium benzoate, ammonium acetate, ammonium formiate, and ammonium lactate, ammonium salts of polymers, and/or their mixtures.

Preferably, from the viewpoint of buffering, the weight ratio of ammonia to ammonium salt(s) in the composition of the present invention is in the range of 10 to 1.

The organic amine(s) and/or salt(s) of organic amines according to the structure above may be selected from mono- and/or diethanolamine, butyl ethanolamine, butyl diethanolamine, dibutyl ethanolamine, methylethanolamine, triethanolamine, N-lauryl diethanolamine, diisopropanolamine, dimethyl isopropanolamine, isopropanolamine, triisopropanolamine, isobutanolamine, triethylamine, tris-(hydroxymethyl)-aminomethane, and/or aminomethyl propanol. Equally suitable are salts of alkyl and/or alkanol amines with a counterion preferably selected from chloride and/or hydrogen chloride, nitrate, sulphate, phosphate, hydrogenphosphate, dihydrogenphosphate, citrate, acetate, sulphite, benzoate, salicylate.

It is preferred from the viewpoint of keratin fibers reshaping performance and keratin fiber damage that the alkalizing agent according to a) is selected from aminomethyl propanol and/or monoethanolamine, and/or diethanolamine and/or tris-(hydroxymethyl)-aminomethane, and/or ammonia, and/or their salts, and/or their mixtures.

The most preferred alkalizing agent according to a) of the composition of the present invention is monoethanolamine, ammonia, and tris-(hydroxymethyl)-aminomethane and/or their/its salt(s).

It is preferred from the viewpoint of providing alkalinity and keratin fibers reshaping performance that the total concentration of alkalizing agent(s) in the composition of the present invention is 0.1% by weight or more, preferably 0.5% by weight or more further more preferably 1% by weight or more, calculated to the total weight of the composition.

It is preferred from the viewpoint of keratin fibers reshaping performance and cosmetic safety that the total concentration of alkalizing agent(s) in the composition of the present invention is 25% by weight or less, preferably 20% by weight or less further more preferably 15% by weight or less, calculated to the total weight of the composition.

For attaining the above-mentioned effects, it is preferred that the total concentration of alkalizing agent(s) of the composition of the present invention is in the range of 0.1% to 25% by weight, preferably in the range of 0.5% to 20% by weight, more preferably in the range of 1% to 15% by weight, calculated to the total weight of the composition.

### Compounds According to b)

The composition of the present invention comprises one or more particulate thickening agent(s) as compound(s) according to group b), which is preferably solid at 25° C. and atmospheric pressure, and partially or completely insoluble in water at 25° C. and atmospheric pressure.

It is preferred from the viewpoint of composition homogeneity that compound(s) according to group b) have a number average particle size in the range of range of 10 nm to 100 μm, preferably in the range of 100 nm to 75 μm, more preferably in the range of 500 nm to 70 μm, still more preferably in the range of 1 μm to 50 μm, determined by static light scattering technique such as by using Malvern Mastersizer offered by Malvern Instruments.

Compound(s) according to group b) may be inorganic, organic, or hybrid inorganic/organic particulate thickening agents.

It is preferred from the viewpoint of commercial availability that compound(s) according to group b) are inorganic particulate thickening agents.

They further preferably are clay minerals, more preferably smectite clay minerals, further more preferably they are smectite clay minerals of natural origin, still further more preferably they are hectorite(s), bentonite(s), montmorrilite(s), kaolin(s), and/or their mixtures.

It is preferred from the viewpoint of formulation freedom that compound(s) according to group b) are synthetic clay minerals, for example synthetic sodium magnesium silicates.

It is preferred from the viewpoint of formulation freedom that compound(s) according to group b) are organic or hybrid organic/inorganic particulate thickening agents.

Organic particulate thickening agents are, for example, starches of natural or partial synthetic origin such as potato starch, rice starch, tapioca starch, and/or cellulose fibers and/or their mixtures.

Hybrid organic/inorganic particulate thickening agents have inorganic and organic moieties. Suitably, they are clay minerals that are modified with cationic surfactants. The resulting hybrid clay minerals have a higher degree of hydrophobicity. Suitable compounds are Quaternium-18 hectorite, Quaternium-18 bentonite, and/or their mixtures.

Suitably, mixtures of inorganic particulate thickening agents or smectite clay minerals of natural origin and hydrophobically-modified clay minerals may be used.

It is preferred from the viewpoint of stabilization that the total concentration of compound(s) according to group b) is 0.1% by weight or more, more preferably 0.5% by weight or more, further more preferably 0.75% by weight or more, still further more preferably 1% by weight or more, calculated to the total weight of the composition.

It is preferred from the viewpoint of economic reasons that the total concentration of compound(s) according to group b) is 30% by weight or less, more preferably 20% by weight or less, further more preferably 15% by weight or less, still further more preferably 10% by weight or less, calculated to the total weight of the composition.

For attaining the above-mentioned effects, it is preferred that that the total concentration of compound(s) according to group b) is in the range of 0.1% to 30% by weight, preferably 0.5% to 20% by weight, more preferably in the range of 0.75% to 15% by weight, more preferably in the range of 1% to 10% by weight, calculated to the total weight of the composition.

### Lipophilic Compounds According to Group c)

The composition of the present invention may comprise one or more lipophilic compound(s) at a total concentration in the range of 1% to 80% by weight, calculated to the total weight of the composition, as compound(s) according to c).

It is preferred from the viewpoint of composition stability that the compound(s) according to group c) are selected from natural and/or vegetable oils, mineral oil, and fatty acid esters consisting of linear or branched, saturated or unsaturated fatty acids with C₁₂ to C₂₂ being esterified with linear or branched primary alcohols with C₃ to C₁₂, silicones, lauryl alcohol, cetearyl alcohol, cetyl alcohol, stearyl alcohol, and/or their mixtures.

Suitable natural and/or vegetable oils are, for example, castor oil, avocado oil, olive oil, sunflower oil, rapeseed oil, wheat germ oil, or almond oil.

Suitable silicones are, for example, linear or cyclic silicones with or without amination, such as dimethicone, trimethicone, and/or amodimethicone.

The preferred compound(s) according to group c) are selected from fatty acid esters consisting of linear or branched, saturated or unsaturated fatty acids with C₁₂ to C₂₂ being esterified with linear or branched primary alcohols with C₃ to C₁₂, more preferably they are selected from isopropyl palmitate, isopropyl laurate, octyl palmitate, isocetyl palmitate, octyl stearate, oleyl oleate, myristyl myristate, and/or their mixtures, form the viewpoint of curling efficiency and composition stability.

The most preferred compound according to c) is isopropyl myristate, isopropyl laurate, and/or cetearyl alcohol, and/or their mixtures, from the viewpoint of keratin fiber reshaping, removability with washing, and composition stability.

It is preferred from the viewpoint of keratin fibers reshaping performance that the total concentration of compound(s) according to group c) is 1% by weight or more, more preferably 2% by weight or more, further more preferably 3% by weight or more, still further more preferably 5% by weight or more, calculated to the total weight of the composition.

It is preferred from the viewpoint of removability with washing and composition stability that the total concentration of compound(s) according to group c) is 80% by weight or less, preferably 50% by weight or less, more preferably 30% by weight or less, further more preferably 20% by weight or less, still further more preferably 15% by weight or less, calculated to the total weight of the composition.

For attaining the above-mentioned effect, it is preferred that the total concentration of compound(s) according to group c) is in the range of 1% to 80% by weight, preferably in the range of 2% to 50% by weight, more preferably in the range of 3% to 30% by weight, further more preferably in the range of 5% to 20% by weight, still further more preferably in the range of 5% to 15% by weight, calculated to the total weight of the composition.

### Thickening Agent as Compound(s) According to Group d)

The composition of the present invention may optionally comprise one or more thickening agent(s) different from the compounds according to group c), as compound(s) according to group d).

Suitably, one or more compound(s) according to group d) is an oil-soluble thickening agent, preferably it is/they are hydrogenated vegetable oil(s), homopolymer(s) or copolymer(s) comprising monomer units of ethylene and/or propylene and/or butylene and/or styrene.

Preferably, the one or more thickening agent(s) according to group d) is/are thickening agents being soluble in or mixable with the compounds according to b).

The term ‘soluble’ within the meaning of the present invention denotes the that the compounds according to d) may be solid at 25° C. and atmospheric pressure, but are able to completely dissolve in the compounds according to b). Preferably, the complete dissolution appears at 25° C. under atmospheric pressure. However, for the purpose of the present invention, it is sufficient that the thickening agent according to d) dissolves in the compounds according to b) at elevated temperatures leading to sufficient thickening of the composition at these temperatures, for example at process temperatures of keratin fiber treatment at 80° C. Hence, it is not required that at 25° C. and atmospheric pressure a complete solution of the compounds according to d) is possible to obtain.

The term ‘mixable’ denotes that the compounds according to d) may be liquid at 25° C. and atmospheric pressure. Hence, the compounds according to d) must be at least partially mixable with the compounds according to b) at 25° C. under atmospheric pressure. However, complete mixability should be obtained at process temperatures of keratin fiber treatment at 80° C., then delivering the required thickening effect.

In one aspect of the present invention, the oil-soluble thickening agent according to d) is an oil-soluble thickening polymer, preferably a non-ionic, anionic, non-ionic, and/or cationic oil-soluble thickening polymer, more preferably a non-ionic oil-soluble polymer.

It is preferred from the viewpoint of manufacturing that the compounds according to d) are solid or pasty at 25° C. under atmospheric conditions.

In one aspect of the present invention, the oil-soluble thickening agent according to d) is an oil-soluble thickening polymer, preferably a non-ionic, anionic, non-ionic, and/or cationic oil-soluble thickening polymer, more preferably a non-ionic oil-soluble polymer.

It is preferred from the viewpoint of oil solubility of the thickening polymer that the compound(s) according to d) is a homopolymer or copolymer comprising monomer units of ethylene and/or propylene and/or butylene and/or styrene.

Suitable non-ionic polymers satisfying the description of above are, for example, copolymers of ethylene/butylene with styrene monomers are commercially available under the trade name Kraton polymers, for example Kraton G1701E.

In another aspect of the present invention, the compounds according to d) is/are hydrogenated vegetable oil, which is commercially available under the trade name Dermofeel Viscolid from Evonik Ind. AG.

Thus, preferred thickening agents according to d) are homopolymers or copolymers comprising monomer units of ethylene and/or propylene and/or butylene and/or styrene and hydrogenated vegetable oil, and/or their mixtures.

The most preferred thickening agents according to d) are homopolymers or copolymers comprising monomer units of ethylene and/or propylene and/or butylene and/or styrene and hydrogenated vegetable oil, and/or their mixtures.

Suitably, one or more compound(s) according to group d) is/are a water-soluble thickening polymer, preferably one or more non-ionic, anionic, cationic, or amphoteric/zwitterionic thickening polymer(s).

Suitable anionic thickening polymers are copolymers and/or crosspolymers which comprise an acrylate and/or methacrylate monomer unit and optionally least one more hydrophobic unit such as alkyl chains. Examples are acrylates/c10-30 alkyl acrylate crosspolymer, acrylates/steareth-20 methacrylate copolymer, acrylates/stearyl acrylate/dimethicone methacrylate copolymer, acrylates/beheneth-25 methacrylate copolymer, acrylates/lauryl acrylate/stearyl acrylate/ethylamine oxide methacrylate copolymer, Hydroxyethyl acrylate/sodium acryloyldimethyl taurate copolymer, carboxymethyl cellulose, alginic acids, sodium alginates, ammonium alginates, calcium alginates, gum arabic, guar gum or xanthan gum, dehydroxanthan gum or acrylic acid polymers known with the CTFA adopted name Carbomer and its derivatives.

Suitable non-ionic thickening polymers are, for example, alkyl modified cellulose derivatives such as (C₂-C₅)-alkylcellulose, cellulose polymers, preferably methyl cellulose, ethyl cellulose, hydroxyethylcellulose, methylhydroxyethylcellulose, methylhydroxypropylcellulose.

Suitable cationic non-associative polymers are Polyquaternium 6, Polyquaternium 16, and Polyquaternium 37.

The most preferred thickening polymers of the composition of the present invention are Carbomer and xanthan gum.

Hence, it is preferred for the purpose of the present invention that the compound(s) according to d) are able to thicken the composition of the present invention at a temperature of 80° C. or more. Thus, it is preferred from the viewpoint of heat stability of the composition and keratin fiber reshaping performance that the viscosity of the composition at 80° C. is 1,000 mPas or more, more preferably 5,000 mPas or more, further more preferably 10,000 mPas or more, measured by plate-cone viscometry, for example with a Brookfield viscometer with appropriate spindle.

It is preferred from the viewpoint of thickening effect and reshaping power that the total concentration of compound(s) according to d) is 0.25% by weight or more, more preferably 0.5% by weight or more, further more preferably 0.5% by weight or more, still further more preferably 1% by weight or more, calculated to the total weight of the composition.

It is preferred from the viewpoint of thickening effect, reshaping power, and product safety that the total concentration of compound(s) according to d) is 0.25% by weight or more, more preferably 0.5% by weight or more, further more preferably 1% by weight or more, calculated to the total weight of the composition.

It is preferred from the viewpoint of thickening effect, reshaping power, and rinsability of keratin fibers that the total concentration of compound(s) according to d) is 20% by weight or less, more preferably 15% by weight or less, further more preferably 12% by weight or less, calculated to the total weight of the composition.

For attaining the above-mentioned effects, it is preferred that the total concentration of compound(s) according to d) is in the range of 0.25% to 20% by weight, preferably in the range of 0.5% to 15% by weight, more preferably 1% to 12% by weight, calculated to the total weight of the composition.

### Surfactants as Compound(s) According to Group e)

The composition of the present invention may comprise one or more surfactant(s) according to group e), preferably selected from non-ionic, anionic, amphoteric and/or zwitterionic, and/or cationic surfactants, and/or their salt(s), and/or their mixtures.

Suitable anionic surfactants are selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures.

Suitable alkyl sulfate or preferably ethoxylated alkyl ether sulfate surfactant or mixtures thereof have an alkyl chain length of C₁₀ to C₂₂.

Suitable example anionic surfactants are laureth sulfates, coceth sulfate, pareth sulfate, capryleth sulphate, myreth sulfate, oleth sulfate, deceth sulfate, trideceth sulfate, coco sulphate, C₁₀-C₁₆ alkyl sulphate, C₁₁-C₁₅ alkyl sulphate, C₁₂-C₁₈ alkyl sulphate, C₁₂-C₁₅ alkyl sulphate, C₁₂-C₁₆ alkyl sulphate, C₁₂-C₁₃ alkyl sulfate, lauryl sulphate, myrystyl sulphate, palm kernel sulphate, cetearyl sulfate, cetyl sulphate, decyl sulphate, oleyl sulphate, behenyl sulphate and/or their salts. All of the aforementioned anionic surfactants may or may not be ethoxylated at various degrees.

Cations for the surfactants may be selected from sodium, potassium, magnesium and/or ammonium.

Suitable non-ionic surfactants are alkyl polyglycosides, ethoxylated triglycerides, ethoxylated fatty alcohols, ethoxylated fatty acid esters, and/or their mixtures.

Preferred non-ionic surfactants are alkyl polyglycosides according to the general structure:

R₂₃O(R₂₄O)ₜZₓ

Wherein Z denotes a carbohydrate with C₅ to C₆, R₂₃ is an alkyl group with Ca to C₁₈, R₂₄ is methyl, ethyl or propyl, t ranges from 0 to 10, and x ranges from 1 to 5. Suitable compounds according to this structure are C₉-C₁₁ alkylpolyglycoside, the structures disclosed in EP-A 70 074, and JP 2015-123019A.

The most preferred compounds according to the structure of above are decyl glucoside, lauryl glucoside, and coco glucoside.

Suitable amphoteric/zwitterionic surfactants are compounds according to the general structure(s)

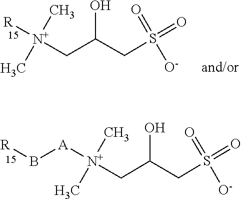

wherein R₁₅ is a straight or branched, saturated or unsaturated, substituted or unsubstituted alkyl chain with a carbon number of C₁₀ to C₂₂, preferably R₁₅ is a straight alkyl chain with a carbon number of C₁₀ to C₁₆, A is a straight alkyl chain with a carbon number of C₁ to C₆ or a branched alkyl chain with a carbon number of C₃ to C₆, preferably A is a linear alkyl chain with a carbon number of C₃, and B is an amide or an ester group.

Suitable compounds are known as hydroxysultaine surfactants, such as cocoamidopropyl hydroxysultaine, laurylamidopropyl hydroxysultaine, erucamidopropyl hydroxysultaine, lauryl hydroxysultaine, and cocoyl hydroxysultaine, and/or their salt(s).

Further suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

The preferred amphoteric/zwitterionic surfactant(s) is/are selected from alkylamido betaines and/or alkylamidoalkyl betaine surfactants.

Suitable cationic surfactants are of quaternary ammonium structure according to the following general structure

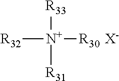

where R₃₀ is a saturated or unsaturated, branched or linear alkyl chain with C₈-C₂₂ or

R₃₄CONH(CH₂)ₙ

where R₃₄ is saturated or unsaturated, branched or linear alkyl chain with C₇-C₂₁ atoms and n has typical value of 1-4 or

R₃₅COO(CH₂)ₙ

where R₃₅ is saturated or unsaturated, branched or linear alkyl chain with C₇-C₂₁ atoms and n has typical value of 1-4, and
R₃₁ is unsaturated or saturated, branched or linear alkyl chain with C₁-C₂₂ atoms or

R₅CONH(CH₂)ₙ

or

R₆COO(CH₂)ₙ

where R₃₄, R₃₅ and n are same as above.

R₃₂ and R₃₃ have an alkyl chain with C₁ to C₄, and X⁻ typically is chloride, bromide, or methosulfate.

Typical examples of those ingredients are cetyl trimethyl ammonium chloride, stearyl trimonium chloride, dipalmitoyl dimonium chloride, distearyl dimethyl ammonium chloride, stearamidopropyl trimethyl ammonium chloride, dioleoylethyl dimethyl ammonium methosulfate, dioleoylethyl hydroxyethylmonium methosulfate, behenyl trimethyl ammonium chloride, and/or their mixtures.

It is preferred from the viewpoint of composition stability that the total concentration of surfactants as compound(s) according to group e) is 0.1% by weight or more, preferably 0.5% by weight or more, further more preferably 0.75% by weight or more, calculated to the total weight of the composition.

It is preferred from the viewpoint of composition stability and cost of goods that the that the total concentration of surfactants as according to group e) is 20% by weight or less, further more preferably 15% by weight or less, still further more preferably 10% by weight or less, calculated to the total weight of the composition.

For attaining the above-mentioned effects, it is preferred that the total concentration of surfactants as compound(s) according to group e) is in the range of 0.1% to 20% by weight, preferably 0.5% to 15% by weight, more preferably in the range of 0.75% to 10% by weight, calculated to the total weight of the composition.

### Process for Reshaping Keratin Fibers

The present invention is also directed to a process for reshaping keratin fibers, preferably human keratin fibers, more preferably human hair comprising the steps of:
i) putting keratin fibers under mechanical tension,
ii) applying to keratin fibers the composition as defined above,
iii) optionally covering keratin fibers with a moisture barrier,
iv) heating the keratin fibers to a temperature in the range of 50° C. to 230° C.,
v) optionally removing the moisture barrier from keratin fibers,
vi) releasing tension from keratin fibers,
vii) optionally rinsing-off the keratin fibers,
wherein process steps i), ii), and vi), vii) can be executed in either order.

It is preferred from the viewpoint of reshaping performance that the process is a permanent waving process.

It is preferred form the viewpoint of user convenience that for process step i) the keratin fibers are put under mechanical tension on a curler or roller having heating means. After completion of step ii) or optionally step iii), the curler may then be connected to a digital perm machine for heating the curler and the keratin fibers.

It is preferred from the viewpoint of hair reshaping performance that the keratin fibers are heated in step iv) to a temperature of 80° C. or more, more preferably to a temperature of 85° C. or more, further more preferably to a temperature of 90° C. or more.

It is preferred from the viewpoint of minimizing damage that the keratin fibers are heated in step iv) to a temperature of 180° C. or less, more preferably to a temperature of 140° C. or less, further more preferably to a temperature of 120° C. or less.

For attaining the above mentioned effect, it is preferred that the keratin fibers are heated in step iv) to a temperature in the range of 80° C. to 180° C., more preferably 85° C. to 140° C., further more preferably 90° C. to 120° C.

It is further preferred from the viewpoint of processing time and speedy hair treatment that the heating time of step iv) is 2 min or more, more preferably 5 min or more, further more preferably 10 min or more, still further more preferably 20 min or more.

It is preferred from the viewpoint of keratin fiber reshaping performance and damage reduction that the heating time of step iv) is 60 min or less, more preferably 45 min or less, still further more preferably 40 min or less.

For attaining the above-mentioned effects, it is preferred that the heating time of step iv) is in the range of 2 min to 60 min, more preferably in the range of 5 min to 45 min, further more preferably in the range of 10 min to 40 min, still further more preferably 20 min to 40 min.

It is preferred from the viewpoint of keratin fiber reshaping performance that in step iii) the keratin fibers are covered with a moisture barrier. The moisture barrier of step iii) may be a foil or wrap impermeable for water vapor, or housing made of a material impermeable for water vapor, with the provision that the selected materials for the moisture barrier are heat resistant up to the selected process temperature.

In principle, many materials are suitable for serving as moisture barrier such as aluminum foil, plastic foil, and/or plastic device, which enclose the curler and/or keratin fiber streak. Alternatively, certain types of anti-flammable fabric is equally suitable. The purpose of the moisture barrier is to keep the keratin fibers moist over the total processing time of heating.

Suitable examples are re-sealable zipper storage bags made of materials such a slow density polyethylene.

For the purpose of the present invention, it is one aspect to cover the keratin fibers with the moisture barrier from the viewpoint of hair reshaping performance. In another aspect from the viewpoint of user convenience, it is preferred that the hair is not covered with a moisture barrier.

### Kit-of-Parts

The present invention is also directed to a a kit-of-parts comprising:
a composition A having a pH in the range of 7 to 12 and comprising one or more compound(s) according to group a) as defined above,
a composition B comprising one or more compound(s) according to group b) as defined above.

The same concentration ranges of compound(s) according to a) and b) are applicable, which have been disclosed above for the reshaping composition. Their respective concentration is calculated to the compositions A or B, respectively.

Preferably, the compositions A and/or B may comprise one or more compound(s) according to group c) and/or d), as also defined for the reshaping composition.

The compositions A and B of the kit are preferably kept separate until directly prior to application onto keratin fibers. The compositions A and B are then mixed and yield the reshaping composition.

The present invention is also directed to a kit-of-parts comprising the reshaping composition as defined above and one or more digital perm curlers(s).

Such kit-of-parts may be offered for sale and have the advantage of user convenience, because it includes composition and devices.

It is further preferred that the kit also comprises one or more moisture barrier for covering the curler as defined in optional step ii) or xiii) above.

Hence, the present invention is also directed to a process for reshaping keratin fibers, preferably human keratin fibers, more preferably human hair comprising the steps of:
x) putting keratin fibers under mechanical tension,
xi) mixing the compositions A and B as defined above to prepare a reshaping composition as defined above,
xii) applying the reshaping composition to keratin fibers,
xiii) optionally covering keratin fibers with a moisture barrier,
xiv) heating the keratin fibers to a temperature in the range of 50° C. to 230° C.,
xv) optionally removing the moisture barrier from keratin fibers,
xvi) releasing tension from keratin fibers,
xvii) optionally rinsing-off the keratin fibers,
wherein process steps x), xi), and xvi), xvii) can be executed in either order.

### Use of Composition

The present invention is also directed to the use of the composition as defined above for permanent reshaping of keratin fibers, preferably of human keratin fibers, more preferably of human hair, still more preferably for permanent waving of human hair.

The following examples are to illustrate the invention, but not to limit it.

### EXAMPLES

Compounds according to a) were dissolved in water. Separately, compounds according to b) were dispersed in compound according to c). The aqueous solution was then heated to 80° C. and the oil phase was added under constant stirring. After cooling the mixture, the pH was adjusted and water balance was added.

Working examples

| | | Comp. | | | | | |
|---|---|---|---|---|---|---|---|
| | | example | Working examples | | | | |
| | | 1 | 2 | 3 | 4 | 5 | 6 |
| | Ingredients | % by weight [active matter] | | | | | |
| a) | Ammonia | 4.0 | 4.0 | 4.0 | 4.0 | 4.0 | 4.0 |
| | (25% v/v) | | | | | | |
| b) | Hectorite | — | 2.0 | | — | 2.0 | 2.0 |
| b) | Kaolin | — | — | 2.0 | — | — | — |
| b) | Bentonite | — | — | — | 2.0 | — | — |
| c) | Isopropyl | 25.0 | 25.0 | 25.0 | 25.0 | 10.0 | 35.0 |
| | myristate | | | | | | |
| — | Coco | 5.0 | 5.0 | 5.0 | 5.0 | 5.0 | 5.0 |
| | glycoside* | | | | | | |
| — | Carbopol 10** | 1.0 | — | — | — | — | — |
| d) | Hydrogenated | — | — | — | — | 1.0 | — |
| | vegetable oil | | | | | | |
| — | NaOH/HCl | Ad pH 9.5 | | | | | |
| — | Water | Ad 100.0 | | | | | |
| Evaluation | Curl ratio L | 9.1 | 10.0 | 11.0 | 10.7 | 10.5 | 11.8 |

*Plantacare 818 sold by BASF Corp.
**Carbopol Ultrez 10 sold by Lubrizol Corp.

### Methods

Human hair streaks (Caucasian, 21 cm long, 2 g per bundle) were purchased from Fischbach+Miller Haar, Laupheim, Germany. The hair streaks were shampooed with a commercially available shampoo under the brand name Goldwell Deep Cleansing Shampoo. Then the streaks were towel dried. Then 1 g of the compositions from above was applied to the hair streaks with a brush. The streaks were then wound on perming rods possessing an electrical heating system. The rods were then heated to a temperature in the range of 90° C. for 20 min with a digital perming machine. Then the rods were allowed to cool down, and the hair was shampooed with the same shampoo from above. The streaks were then blow-dried.

Assessment of curling efficiency was investigated by measuring and calculating the curl ratio L according to the formula:

L=(L₀−Lₜ)/L₀

wherein L₀ is the length of the hair streak prior to curling and Lₜ is the length of the hair streak after the curling experiment. The number is reported as percentage and a higher percentage corresponds to higher curling degree.

| | | Comp. | | | |
|---|---|---|---|---|---|
| | | example | Working examples | | |
| | | 7 | 8 | 9 | 10 |
| | Ingredients | % by weight [active matter] | | | |
| a) | Monoethanolamine | 0.5 | 0.5 | 0.5 | 0.5 |
| a) | Ammonia (25% v/v) | 0.5 | 0.5 | 0.5 | 0.5 |
| a) | Tris-(hydroxymethyl)- | 5.0 | 5.0 | 5.0 | 5.0 |
| | aminomethane | | | | |
| b) | Hectorite | — | 2.0 | — | — |
| c) | Cetearyl alcohol | 5.0 | 5.0 | 5.0 | 5.0 |
| — | Ceteareth-30 | 2.5 | 2.5 | 2.5 | 2.5 |
| c) | Kaolin | — | — | 2.0 | — |
| c) | Bentonite | — | — | — | 2.0 |
| — | NaOH/HCl | Ad pH 9.5 | | | |
| — | Water | Ad 100.0 | | | |
| Evaluation | Curl ratio L | 6.1 | 11.2 | 10.8 | 10.5 |

The same methods have been used as described above.

1. A cosmetic composition for reshaping keratin fibers with heat having a pH in the range of 7 to 12, and comprising:
a) one or more alkalizing agent(s) selected from
ammonia and/or ammonium salt(s),
organic amine(s) and/or salt(s) of organic amines according to the following general structure: 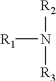
wherein R₁, R₂, and R₃ are independently selected from H, linear C₁-C₆ alkyl which may be substituted with one hydroxyl group, or branched C₃-C₁₂ alkyl or alkanol, wherein at least one of R₁, R₂, or R₃ is different from H, and/or
a mixture thereof;
b) one or more particulate thickening agent(s),
wherein the cosmetic composition comprises less than 1% by weight of oxidizing agents, calculated to a total weight of the cosmetic composition.

2. The cosmetic composition of claim 1, wherein the cosmetic composition comprises less than 1% by weight of reducing agents, calculated to the total weight of the cosmetic composition.

3. (canceled)

4. The cosmetic composition of claim 1, wherein the pH of the cosmetic composition is in the range of 8.0 to 10.5.

5. (canceled)

6. (canceled)

7. The cosmetic composition of claim 1, wherein one or more compound(s) according to b) have a number average particle size in the range of 10 nm to 100 μm, determined by static light scattering technique.

8. The cosmetic composition of claim 1, wherein one or more compound(s) according to b) is/are inorganic, organic or hybrid inorganic/organic particulate thickening agents.

9. The cosmetic composition of claim 1, wherein one or more compound(s) according to b) are inorganic particulate thickening agent(s).

10. The cosmetic composition of claim 1, wherein one or more compound(s) according to b) is/are organic particulate thickening agent(s).

11. (canceled)

12. The cosmetic composition of claim 1, wherein a total concentration of one or more compound(s) according to b) is in the range of 0.1% to 30% by weight, calculated to the total weight of the cosmetic composition.

13. The cosmetic composition of claim 1, wherein the cosmetic composition further comprises one or more lipophilic compound(s) as compound(s) according to c) selected from natural and/or vegetable oils, mineral oil, and fatty acid esters consisting of linear or branched, saturated or unsaturated fatty acids with C₁₂ to C₂₂ being esterified with linear or branched Primary alcohols with C₃ to C₁₂, and/or silicones, and/or lauryl alcohol, cetearyl alcohol, cetyl alcohol, stearyl alcohol, and/or their mixtures.

14. (canceled)

15. The cosmetic composition of claim 13, wherein a total concentration of the one or more lipophilic compound(s) according to c) is in the range of 1% to 80% by weight, calculated to the total weight of the composition.

16. (canceled)

17. The cosmetic composition of claim 1, wherein the cosmetic composition is a permanent waving composition.

18. A process for reshaping keratin fibers comprising:
i) putting keratin fibers under mechanical tension,
ii) applying to keratin fibers the cosmetic composition of claim 1,
iii) optionally covering keratin fibers with a moisture barrier,
iv) heating the keratin fibers to a temperature in the range of 50° C. to 230° C.,
v) optionally removing the moisture barrier from keratin fibers,
vi) releasing tension from keratin fibers,
vii) optionally rinsing-off the keratin fibers,
wherein process steps i), ii), and vi), vii) can be executed in either order.

19. A kit-of-parts comprising
a composition A having a pH in the range of 7 to 12 and comprising one or more compound(s) according to a) as defined in claim 1,
a composition B comprising one or more compound(s) according to b) as defined in claim 1.

20. A kit-of-parts comprising the cosmetic of claim 1 and one or more digital perm curler(s).

21. (canceled)

22. The cosmetic composition of claim 1, wherein one or more compound(s) according to b) is/are clay minerals.

23. The cosmetic composition of claim 1, wherein one or more compound(s) according to b) is/are smectite clay minerals.

24. The cosmetic composition of claim 1, wherein one or more compound(s) according to b) is/are hectorite(s), bentonite(s), kaolin(s), montmorrilite(s), and/or a mixture thereof.

25. The cosmetic composition of claim 1, wherein one or more compound(s) according to b) is/are starches of natural or partial synthetic origin selected from at least one of potato starch, rice starch, tapioca starch, cellulose fibers, and/or a mixture thereof.

26. The cosmetic composition of claim 1, wherein one or more compound(s) according to b) is/are hydrophobically-modified clay minerals.

27. The composition of claim 1, wherein one or more compound(s) according to b) is/are Quaternium-18 hectorite, Quaternium-18 bentonite, and/or a mixture thereof.
